(19) United States
(12) Patent Application Publication
(10) Pub. No.: US 2003/0166528 A1
(43) Pub. Date: Sep. 4, 2003

(54) Antifungal compound

(76) Inventor: Jong-Jye Lin, Hercules, CA (US)

Correspondence Address: MARSHALL, GERSTEIN & BORUN, 6300 SEARS TOWER, 233 SOUTH WACKER, CHICAGO, IL, 60606-6357, US

(21) Appl. No.: 09/975,638

(22) Filed: Oct. 11, 2001

Related U.S. Application Data
(60) Provisional application No. 60/239,709, filed on Oct. 11, 2000.

(51) Int. Cl. A61K 38/16; C07K 14/35

(52) U.S. Cl. 514/12; 530/350

(57) ABSTRACT

The present invention relates generally to a novel peptide, XMP.676, derived from or based on Domain III (amino acids 142-169) of bactericidal/permeability-increasing protein (BPI) and therapeutic uses of this peptide.

This application claims benefit on U.S. Provisional Application 60/239,709 filed Oct. 11, 2000.

### FIELD OF THE INVENTION

The present invention relates generally to peptide constructs derived from or based on Domain III (amino acids 142-169) of bactericidal/permeability-increasing protein (BPI) and therapeutic uses of such peptides.

### BACKGROUND OF THE INVENTION

Infectious diseases can be caused by a number of organisms, including bacteria, fungi, protozoans and other parasites, and viruses. Bacteria as a group generally include gram-negative bacteria, gram-positive bacteria, spirochetes, rickettsiae, mycoplasmas, mycobacteria and actinomycetes. Resistance of bacteria and other pathogenic organisms to antimicrobial agents is an increasingly troublesome problem. The accelerating development of antibiotic-resistant bacteria, intensified by the widespread use of antibiotics in farm animals and overprescription of antibiotics by physicians, has been accompanied by declining research into new antibiotics with different modes of action. [Science, 264: 360-374 (1994).]

Fungi are eukaryotic cells that may reproduce sexually or asexually and may be dimorphic. Fungi are not only important human and animal pathogens, but they are also among the most common causes of plant disease. Fungal infections (mycoses) are becoming a major concern for a number of reasons, including the limited number of antifungal agents available, the increasing incidence of species resistant to known antifungal agents, and the growing population of immunocompromised patients at risk for opportunistic fungal infections, such as organ transplant patients, cancer patients undergoing chemotherapy, burn patients, AIDS patients, or patients with diabetic ketoacidosis. The incidence of systemic fungal infections increased 600% in teaching hospitals and 220% in non-teaching hospitals during the 1980's. The most common clinical isolate is Candida albicans (comprising about 19% of all isolates). In one study, nearly 40% ofall deaths from hospital-acquired infections were due to fungi. [Sternberg, Science, 266:1632-1634 (1994).].

Newly explored as antifungal agents are a class of products related to bactericidal/permeability-increasing protein (BPI). BPI is a protein isolated from the granules of mammalian polymorphonuclear leukocytes (PMNs or neutrophils), which are blood cells essential in the defense against invading microorganisms. The amino acid sequence of the entire human BPI protein and the nucleic acid sequence of DNA encoding the protein have been reported in FIG. 1 (SEQ ID NOS: 2 and 3) of Gray et al., J. Biol. Chem., 264:9505 (1989), incorporated herein by reference. Recombinant human BPI holoprotein has also been produced in which valine at position 151 is specified by GTG rather than GTC, residue 185 is glutamic acid (specified by GAG) rather than lysine (specified by AAG) and residue 417 is alanine (specified by GCT) rather than valine (specified by GTT). See U.S. Pat. No. 5,627,153.

Three separate functional domains within the recombinant 23 kD N-terminal BPI sequence have been discovered (Little et al., 1994, J. Biol. Chem. 269: 1865). These functional domains of BPI designate regions of the amino acid sequence of BPI that contribute to the total biological activity of the protein and were essentially defined by the activities of proteolytic cleavage fragments, overlapping 15-mer peptides and other synthetic peptides. Domain I is defined as the amino acid sequence of BPI comprising from about amino acid 17 to about amino acid 45. Initial peptides based on this domain were moderately active in both the inhibition of LPS-induced LAL activity and in heparin binding assays, and did not exhibit significant bactericidal activity. Domain II is defined as the amino acid sequence of BPI comprising from about amino acid 65 to about amino acid 99. Initial peptides based on this domain exhibited high LPS and heparin binding capacity and exhibited significant antibacterial activity. Domain III is defined as the amino acid sequence of BPI comprising from about amino acid 142 to about amino acid 169. Initial peptides based on this domain exhibited high LPS and heparin binding activity and exhibited surprising antimicrobial activity, including antifungal and antibacterial (including, e.g., anti-gram-positive and anti-gram-negative) activity. The biological activities of peptides derived from or based on these functional domains (i.e., functional domain peptides) may include LPS binding, LPS neutralization, heparin binding, heparin neutralization or antimicrobial activity, including antifungal and/or antibacterial (including e.g., anti-gram-positive and/or anti-gram-negative) activity.

BPI protein products are described in U.S. Pat. No. 5,627,153 and corresponding International Publication No. WO 95/19179 (PCT/US95/00498), all of which are incorporated by reference herein, to have antifungal activity. BPI-derived antifungal peptides are described in co-owned, co-pending U.S. application Ser. No. 08/621,259 filed Mar. 21, 1996, now U.S. Pat. No. 5,858,974, which is in turn a continuation-in-part of U.S. application Ser. No. 08/504,841 filed Jul. 20, 1994 and corresponding International Publication Nos. WO 96/08509 (PCT/US95/09262) and WO 97/04008 (PCT/US96/03845), all of which are incorporated by reference herein. Other peptides with antifungal activity are described in U.S. Pat. No. 5,652,332 [corresponding to International Publication No. WO 95/19372 (PCT/US94/10427)], and in U.S. Pat. Nos. 5,763,567 and 5,733,872 [corresponding to International Publication No. WO 94/20532 (PCT/US94/02465)], which is a continuation-in-part of U.S. patent application Ser. No. 08/183,222 filed Jan. 14, 1994, which is a continuation-in-part of U.S. patent application Ser. No. 08/093,202 filed Jul. 15, 1993 [corresponding to International Publication No. WO 94/20128 (PCT/US94/02401)], which is a continuation-in-part of U.S. patent application Ser. No. 08/030,644 filed Mar. 12, 1993, now U.S. Pat. No. 5,348,942, the disclosures of all of which are incorporated herein by reference. Peptide constructs comprising peptides with antifungal activity that are modified with hydrophobic moieties are described in U.S. Ser. No. 09/602,847 filed Jun. 23, 2000 and International Publication No. WO ______ (Int'l Application No. PCT/US00/17383), the disclosures of all of which are incorporated herein by reference.

BPI protein products exhibit antifungal activity, and enhance the activity of other antifungal agents, as described in U.S. Pat. No. 5,627,153 and International Publication No. WO 95/19179 (PCT/US95/00498), and further as described for BPI-derived peptides in U.S. Pat. No. 5,858,974, which is in turn a continuation-in-part of U.S. application Ser. No. 08/504,841 and corresponding International Publication Nos. WO 96/08509 (PCT/US95/09262) and WO 97/04008 (PCT/US96/03845), as well as in U.S. Pat. Nos. 5,733,872, 5,763,567, 5,652,332, 5,856,438 and corresponding International Publication Nos. WO 94/20532 (PCT/US/94/02465) and WO 95/19372 (PCT/US94/10427).

Gram-positive bacteria have a typical lipid bilayer cytoplasmic membrane surrounded by a rigid cell wall that gives the organisms their characteristic shape, differentiates them from eukaryotic cells, and allows them to survive in osmotically unfavorable environments. This cell wall is composed mainly of peptidoglycan, a polymer of N-acetylglucosamine and N-acetylmuramic acid. In addition, the cell walls of gram-positive bacteria contain teichoic acids which are anchored to the cytoplasmic membrane through lipid tails, giving rise to lipoteichoic acids. The various substituents on teichoic acids are often responsible for the biologic and immunologic properties associated with disease due to pathogenic gram-positive bacteria. Most pathogenic gram-positive bacteria have additional extracellular structures, including surface polysaccharides, capsular polysaccharides, surface proteins and polypeptide capsules.

Gram-negative bacteria also have a cytoplasmic membrane and a peptidoglycan layer similar to but reduced from that found in gram-positive organisms. However, gram-negative bacteria have an additional outer membrane that is covalently linked to the tetrapeptides of the peptidoglycan layer by a lipoprotein; this protein also contains a special lipid substituent on the terminal cysteine that embeds the lipoprotein in the outer membrane. The outer layer of the outer membrane contains the lipopolysaccharide (LPS) constituent.

BPI protein products are bactericidal for gram-negative bacteria, as described in U.S. Pat. Nos. 5,198,541, 5,641,874, 5,948,408, 5,980,897 and 5,523,288. International Publication No. WO 94/20130 proposes methods for treating subjects suffering from an infection (e.g. gastrointestinal) with a species from the gram-negative bacterial genus Helicobacter with BPI protein products. BPI protein products also enhance the effectiveness of antibiotic therapy in gram-negative bacterial infections, as described in U.S. Pat. Nos. 5,948,408, 5,980,897 and 5,523,288 and International Publication Nos. WO 89/01486 (PCT/US99/02700) and WO 95/08344 (PCT/US94/11255). BPI protein products are also bactericidal for gram-positive bacteria and mycoplasma, and enhance the effectiveness of antibiotics in gram-positive bacterial infections, as described in U.S. Pat. Nos. 5,578,572 and 5,783,561 and International Publication No. WO 95/19180 (PCT/US95/00656). BPI protein products exhibit anti-protozoan activity, as described in U.S. Pat. Nos. 5,646,114 and 6,013,629 and International Publication No. WO 96/01647 (PCT/US95/08624). BPI protein products exhibit anti-chlamydial activity, as described in co-owned U.S. Pat. No. 5,888,973 and WO 98/06415 (PCT/US97/13810). Finally, BPI protein products exhibit anti-mycobacterial activity, as described in co-owned, co-pending U.S. application Ser. No. 08/626,646, which is in turn a continuation of U.S. application Ser. No. 08/285,803, which is in turn a continuation-in-part of U.S. application Ser. No. 08/031,145 and corresponding International Publication No. WO 94/20129 (PCT/US94/02463).

Many other utilities of BPI protein products, including rBPI₂₃ and rBPI₂₁, have been described due to the wide variety of biological activities of these products. The effects of BPI protein products in humans with endotoxin in circulation, including effects on TNF, IL-6 and endotoxin are described in U.S. Pat. Nos. 5,643,875, 5,753,620 and 5,952,302 and corresponding International Publication No. WO 95/19784 (PCT/US95/01151).

BPI protein products are also useful for treatment of specific disease conditions, such as meningococcemia in humans (as described in U.S. Pat. Nos. 5,888,977 and 5,990,086 and International Publication No. WO97/42966 (PCT/US97/08016), hemorrhage due to trauma in humans, (as described in U.S. Pat. Nos. 5,756,464 and 5,945,399, U.S. application Ser. No. 08/862,785 and corresponding International Publication No. WO 97/44056 (PCT/US97/08941), burn injury (as described in U.S. Pat. No. 5,494,896 and corresponding International Publication No. WO 96/30037 (PCT/US96/02349)) ischemia/reperfusion injury (as described in U.S. Pat. No. 5,578,568), and depressed RES/liver resection (as described in co-owned, co-pending U.S. application Ser. No. 08/582,230 which is in turn a continuation of U.S. application Ser. No. 08/318,357, which is in turn a continuation-in-part of U.S. application Ser. No. 08/132,510, and corresponding International Publication No. WO 95/10297 (PCT/US94/11404).

BPI protein products also neutralize the anticoagulant activity of exogenous heparin, as described in U.S. Pat. No. 5,348,942, neutralize heparin in vitro as described in U.S. Pat. No. 5,854,214, and are useful for treating chronic inflammatory diseases such as rheumatoid and reactive arthritis, for inhibiting endothelial cell proliferation, and for inhibiting angiogenesis and for treating angiogenesis-associated disorders including malignant tumors, ocular retinopathy and endometriosis, as described in U.S. Pat. Nos. 5,639,727, 5,807,818 and 5,837,678 and International Publication No. WO 94/20128 (PCT/US94/02401).

BPI protein products are also useful in antithrombotic methods, as described in U.S. Pat. Nos. 5,741,779 and 5,935,930 and corresponding International Publication No. WO 97/42967 (PCT/US7/08017).

There continues to exist a need in the art for new antimicrobial compounds, including, for example, those that target animal and plant pathogens. In particular, effective antifungal therapy for systemic mycoses is limited. Products and methods responsive to this need would ideally involve substantially non-toxic compounds available in large quantities by means of synthetic or recombinant methods. Ideal therapeutic compounds would have a rapid effect following systemic and/or oral administration, high potency, low toxicity, and a broad spectrum of activity against a variety of different microbes when administered or applied as the sole agent. Ideal compounds would also be useful in combinative therapies with other antimicrobial agents, particularly where these activities would reduce the amount of other antimicrobial agent required for therapeutic effectiveness, enhance the effect of such agents, or limit potential toxic responses and high cost of treatment.

### SUMMARY OF THE INVENTION

The present invention provides an antifungal peptide construct comprising the amino acid sequence set forth in SEQ ID NO: 1 and therapeutic uses of this construct. A construct according to the present invention includes a novel peptide, designated XMP.676, derived from or based on Domain III (amino acids 142-169) of bactericidal/permeability-increasing protein (BPI) and therapeutic uses of this peptide, especially as an antifungal agent. The sequence of XMP.676 is set forth in SEQ ID NO: 1. A variety of peptide constructs comprising this peptide, e.g., involving addition, deletion or substitution of other amino acid moieties, addition of one or more terminal or internal hydrophobic moieties (e.g., derivative constructs), including sequences with similar biological properties or activities, are also contemplated according to the invention.

An exemplary peptide construct, XMP.676, exhibits potent fungicidal activity against a broad range of fungal species and is surprisingly effective against both Candida and Aspergillus, in vitro and in vivo. Peptide constructs according to the invention not only exhibit excellent antifungal activity but also exhibit other biological activities of BPI protein products, including other antimicrobial activities (e.g., activity against gram-positive bacteria, gram-negative bacteria, chlamydia, prokaryotes, protozoa or other parasites) and endotoxin neutralization activity. Such peptide constructs may also exhibit heparin binding and/or neutralization activity. Consequently, peptide constructs according to the invention are expected to be useful in any of the uses described herein or known in the art for BPI protein products.

Peptide constructs, or pharmaceutical compositions comprising such constructs and suitable diluents, adjuvants or carriers, may be administered alone or concurrently with other known antimicrobial (particularly antifungal) agents. When used as adjunctive therapy, the peptide construct may reduce the amount of the other agent needed for effective therapy, enhance the effect of such other agent, accelerate the effect of such other agent, or reverse (e.g., overcome) resistance of the pathogenic organism to such other agent. The peptide construct may be effective for treating animals (e.g., mammals) in vivo, for treating plants, and for a variety of in vitro uses such as to decontaminate fluids or surfaces or to decontaminate surgical or other medical equipment or implantable devices, including prosthetic joints or indwelling invasive devices.

A further aspect of the invention involves use of the peptide construct for the manufacture of a medicament for treatment of microbial infection, e.g., fungal or bacterial infection. The medicament may additionally include other chemotherapeutic agents such as antimicrobial agents.

Numerous additional aspects and advantages of the invention will become apparent to those skilled in the art upon considering the following detailed description of the invention, which describes the presently preferred embodiments thereof.

### BRIEF DESCRIPTION OF THE DRAWINGS

FIG. 1 displays mortality data from a study of effects of XMP.676 in a mouse candidiasis model using a rapid bolus injection.

FIG. 2 displays results from a similar study in which XMP.676 was administered via slow injection versus a rapid bolus injection.

FIG. 3 displays mortality data from a study of effects of XMP.676 in an immunosuppressed mouse aspergillosis model.

FIG. 4 shows antifungal activity of XMP.676 when incubated with media, mouse serum or human serum.

### DETAILED DESCRIPTION OF THE INVENTION

The present invention provides an antifungal peptide construct comprising the amino acid sequence set forth in SEQ ID NO: 1 and its therapeutic uses, including a novel peptide designated XMP.676 (SEQ ID NO: 1) having the following sequence (listed left to right from the N-terminus to the C-terminus):

Lys_{D}-(Naph-1-Ala)-Norvaline-Ile-Gln-Norvaline-Val-(Thienyl-Ala)-Lys_{D}-Lys_{D} (wherein amino acids labeled with a subscript D are D-amino acids and the remainder are L-amino acids). XMP.676 has been demonstrated to possess antifungal activity in a variety of in vitro killing assays or in vivo models of fungal infection, including, for example, by measuring improved host survival or a reduction of colony-forming units in organs after fungal challenge. Other properties of XMP.676, such as serum stability, were also determined. XMP.676 was observed to exhibit a spectrum of superior properties, including consistent high potency with a broad therapeutic range in vivo, in one or more of the preceding assays in comparison to other peptides derived from or based on Domain III of BPI (“Domain III derived peptides”).

The invention also provides methods of using peptide constructs such as XMP.676 for treating a subject suffering from infection (including fungal, bacterial, or other microbial infection), especially mammalian subjects such as humans, but also including farm animals such as cows, sheep, pigs, horses, goats and/or poultry (e.g., chickens, turkeys, ducks and/or geese), companion animals such as dogs and/or cats, exotic and/or zoo animals, and/or laboratory animals including mice, rats, rabbits, guinea pigs, and/or hamsters. Immunocompromised or immunosuppressed subjects, e.g., subjects suffering from cancer, subjects undergoing radiation therapy and/or cytotoxic chemotherapy, subjects being treated with immunosuppressive drugs, and/or subjects suffering from natural or acquired immune deficiencies such as AIDS, may be treated according to this aspect of the invention. Treatment of infection of plants is also contemplated. “Treatment” as used herein encompasses both prophylactic and/or therapeutic treatment, and may be accompanied by concurrent administration of other antimicrobial agents, including any of the agents discussed herein.

Fungal infection that may be treated according to the invention may be caused by a variety of fungal species including Candida (including C. albicans, C. tropicalis, C. parapsilosis, C. stellatoidea, C. krusei, C. parakrusei, C. lusitanae, C. pseudotropicalis, C. guilliermondi, C. dubliniensis, C. famata or C. glabrata), Aspergillus (including A. fumigatus, A. flavus, A. niger, A. nidulans, A. terreus, A. sydowi, A. flavatus, or A. glaucus), Cryptococcus, Histoplasma, Coccidioides, Paracoccidioides, Blastomyces, Basidiobolus, Conidiobolus, Rhizopus, Rhizomucor, Mucor, Absidia, Mortierella, Cunninghamella, Saksenaea, Pseudallescheria, Paecilomyces, Fusarium, Trichophyton, Trichosporon, Microsporum, Epidermophyton, Scytalidium, Malassezia, Actinomycetes, Sporothrix, Penicillium, Saccharomyces or Pneumocystis.

Other infections that may be treated using a peptide construct according to the invention may be caused by gram-negative bacterial species that include Acidaminococcus, Acinetobacter, Aeromonas, Alcaligenes, Bacteroides, Bordetella, Branhamella, Brucella, Burkholderia, Calymmatobacterium, Campylobacter, Cardiobacterium, Chromobacterium, Citrobacter, Edwardsiella, Enterobacter, Escherichia, Flavobacterium, Francisella, Fusobacterium, Haemophilus, Klebsiella, Legionella, Moraxella, Morganella, Neisseria, Pasturella, Plesiomonas, Porphyromonas, Prevotella, Proteus, Providencia, Pseudomonas, Salmonella, Serratia, Shigella, Stentrophomonas, Streptobacillus, Treponema, Veillonella, Vibrio, or Yersinia species; Chlamydia; or gram-positive bacterial species that include Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Nocardia, Actinomyces, or Corynebacterium species as well as Mycoplasma, Ureaplasma, or Mycobacteria.

Other infections include infections by protozoa including Plasmodia, Toxoplasma, Leishmania, Trypanosoma, Giardia, Entamoeba, Acanthamoeba, Nagleria, Hartmanella, Balantidium, Babesia, Cryptosporidium, Isospora, Microsporidium, Trichomonas or Pneumocystis species; or infections by other parasites include helminths.

Other therapeutic uses of peptide constructs such as XMP.676 according to the invention include methods of treating conditions associated with endotoxin, such as exposure to gram-negative bacterial endotoxin in circulation, endotoxemia, bacterial and/or endotoxin-related shock and one or more conditions associated therewith, including a systemic inflammatory response, cytokine overstimulation, complement activation, disseminated intravascular coagulation, increased vascular permeability, anemia, thrombocytopenia, leukopenia, pulmonary edema, adult respiratory distress syndrome, renal insufficiency and failure, hypotension, fever, tachycardia, tachypnea, and metabolic acidosis. Thus, not only gram-negative bacterial infection but also conditions which are associated with exposure to gram-negative bacterial endotoxin (infection-related conditions) may be ameliorated through endotoxin-binding or endotoxin-neutralizing activities of peptide constructs such as XMP.676.

Therapeutic compositions of the peptide construct may include a pharmaceutically acceptable diluent, adjuvant, or carrier. The peptide construct may be administered without or in conjunction with known surfactants, other chemotherapeutic agents or additional known antimicrobial agents. As described in U.S. application Ser. No. 08/586,133 filed Jan. 12, 1996, which is in turn a continuation-in-part of U.S. application Ser. No. 08/530,599 filed Sep. 19, 1995, which is in turn a continuation-in-part of U.S. application Ser. No. 08/372,104 filed Jan. 13, 1995, and corresponding International Publication No. WO96/21436 (PCT/US96/01095), all of which are incorporated herein by reference, other poloxamer formulations with enhanced activity may be utilized.

Compositions, including therapeutic compositions, of the peptide construct of the invention may be administered systemically or topically. Systemic routes of administration include oral, intravenous, intramuscular or subcutaneous injection (including into depots for long-term release), intraocular or retrobulbar, intrathecal, intraperitoneal (e.g. by intraperitoneal lavage), intrapulmonary (using powdered drug, or an aerosolized or nebulized drug solution), or transdermal. Topical routes include administration in the form of rinses, washes, salves, creams, jellies, drops or ointments (including opthalmic and otic preparations), suppositories, such as vaginal suppositories, or irrigation fluids (for, e.g., irrigation of wounds).

Suitable dosages include doses ranging from 1 μg/kg to 100 mg/kg per day and doses ranging from 0.1 mg/kg to 20 mg/kg per day. When given parenterally, compositions are generally injected in one or more doses ranging from 1 mg/kg to 100 mg/kg per day, preferably at doses ranging from 0.1 mg/kg to 20 mg/kg per day, and more preferably at doses ranging from 1 to 20 mg/kg/day. As described herein for compositions with XMP.676, parenteral doses of 0.5 to 5 mg/kg/day are preferred according to the present invention. The treatment may continue by continuous infusion or intermittent injection or infusion, or a combination thereof, at the same, reduced or increased dose per day for as long as determined by the treating physician. When given topically, compositions are generally applied in unit doses ranging from 1 mg/mL to 1 gm/mL, and preferably in doses ranging from 1 mg/mL to 100 mg/mL. Decontaminating doses are applied including, for example, for fluids or surfaces or to decontaminate or sterilize surgical or other medical equipment or implantable devices, including, for example prosthetic joints or in indwelling invasive devices. Those skilled in the art can readily optimize effective dosages and administration regimens for therapeutic, including decontaminating, compositions as determined by good medical practice and the clinical condition of the individual subject.

“Concurrent administration,” or “co-administration,” as used herein includes administration of one or more agents, in conjunction or combination, together, or before or after each other. The agents maybe administered by the same or by different routes. If administered via the same route, the agents may be given simultaneously or sequentially, as long as they are given in a manner sufficient to allow all agents to achieve effective concentrations at the site of action. For example, a peptide construct may be administered intravenously while the second agent(s) is(are) administered intravenously, intramuscularly, subcutaneously, orally or intraperitoneally. A peptide construct and a second agent(s) may be given sequentially in the same intravenous line or may be given in different intravenous lines. Alternatively, a peptide construct may be administered in a special form for gastric or aerosol delivery, while the second agent(s) is(are) administered, e.g., orally.

Concurrent administration of the peptide construct of the invention, such as XMP.676, for adjunctive therapy with one or more other antimicrobial agents (particularly antifungal agents) is expected to improve the therapeutic effectiveness of the antimicrobial agents. This may occur through reducing the concentration of antimicrobial agent required to eradicate or inhibit target cell growth, e.g., replication. Because the use of some antimicrobial agents is limited by their systemic toxicity or prohibitive cost, lowering the concentration of antimicrobial agent required for therapeutic effectiveness reduces toxicity and/or cost of treatment, and thus allows wider use of the agent. For example, concurrent administration of the peptide construct, such as XMP.676 peptide, and another antifungal agent may produce a more rapid or complete fungicidal or fungistatic effect than could be achieved with either agent alone. Administration of the peptide construct, such as XMP.676 peptide, may reverse the resistance of fungi to antifungal agents or may convert a fungistatic agent into a fungicidal agent. Similar results may be observed upon concurrent administration of the peptide construct, such as XMP.676, with other antimicrobial agents, including antibacterial and/or anti-endotoxin agents.

Therapeutic effectiveness in vivo is based on a successful clinical outcome, and does not require that the antimicrobial agent or agents kill 100% of the organisms involved in the infection. Success depends on achieving a level of antimicrobial activity at the site of infection that is sufficient to inhibit growth or replication of the pathogenic organism in a manner that tips the balance in favor of the host. When host defenses are maximally effective, the antimicrobial effect required may be minimal. Reducing organism load by even one log (a factor of 10) may permit the host's own defenses to control the infection. In addition, augmenting an early microbicidal/microbistatic effect can be more important than a long-term effect. These early events are a significant and critical part of therapeutic success, because they allow time for host defense mechanisms to activate.

In addition, the invention provides a method of killing or inhibiting growth of pathogenic organisms (particularly fungi) comprising contacting the organism with the peptide construct, such as XMP.676, optionally in conjunction with other antimicrobial agents. This method can be practiced in vivo, ex vivo, or in a variety of in vitro uses such as to decontaminate fluids or surfaces or to sterilize surgical or other medical equipment or implantable devices, including prostheses orintrauterine devices. These methods can also be used for in situ decontamination and/or sterilization of indwelling invasive devices such as intravenous lines and catheters, which are often foci of infection.

A further aspect of the invention involves use of the peptide construct, such as XMP.676, for the manufacture of a medicament for treatment of microbial infection (e.g., fungal or bacterial infection) or a medicament for concurrent administration with another agent for treatment of microbial infection. The medicament may optionally comprise a pharmaceutically acceptable diluent, adjuvant or carrier and also may include, in addition to the peptide construct (such as XMP.676), other chemotherapeutic agents.

Known antifungal agents which can be co-administered or combined with the peptide construct, such as XMP.676, according to the invention include polyene derivatives, such as amphotericin B (including lipid or liposomal formulations thereof) or the structurally related compounds nystatin or pimaricin; flucytosine (5-fluorocytosine); azole derivatives (including ketoconazole, clotrimazole, miconazole, econazole, butoconazole, oxiconazole, sulconazole, tioconazole, terconazole, fluconazole, itraconazole, voriconazole [Pfizer], posaconazole [SCH56592, Schering-Plough]) or ravuconazole [Bristol-Myers Squibb]; allylamines-thiocarbamates (including tolnaftate, naftifine or terbinafine); griseofulvin; ciclopirox; haloprogin; echinocandins (including caspofungin [MK-0991, Merck], FK463 [Fujisawa], cilofungin [Eli Lilly] or VER-002 [Versicor]); nikkomycins; or sordarins. Recently discovered as antifungal agents are a class of products based on or derived from bactericidal/permeability-increasing protein (BPI), described in U.S. Pat. Nos. 5,627,153, 5,858,974, 5,652,332, 5,856,438, 5,763,567 and 5,733,872, the disclosures of all of which are incorporated herein by reference.

The polyene derivatives, which include amphotericin B or the structurally related compounds nystatin or pimaricin, are broad-spectrum antifungals that bind to ergosterol, a component of fungal cell membranes, and thereby disrupt the membranes. Amphotericin B is usually effective for systemic mycoses, but its administration is limited by toxic effects that include fever, kidney damage, or other accompanying side effects such as anemia, low blood pressure, headache, nausea, vomiting or phlebitis. The unrelated antifungal agent flucytosine (5-fluorocytosine), an orally absorbed drug, is frequently used as an adjunct to amphotericin B treatment for some forms of candidiasis or cryptococcal meningitis. Its adverse effects include bone marrow depression, including with leukopenia or thrombocytopenia.

The azole derivatives impair synthesis of ergosterol and lead to accumulation of metabolites that disrupt the function of fungal membrane-bound enzyme systems (e.g., cytochrome P450) and inhibit fungal growth. This group of agents includes ketoconazole, clotrimazole, miconazole, econazole, butoconazole, oxiconazole, sulconazole, tioconazole, terconazole, fluconazole or itraconazole. Significant inhibition of mammalian P450 results in significant drug interactions. Some of these agents may be administered to treat systemic mycoses. Ketoconazole, an orally administered imidazole, is used to treat nonmeningeal blastomycosis, histoplasmosis, coccidioidomycosis or paracoccidioidomycosis in non-immunocompromised patients, and is also useful for oral and esophageal candidiasis. Adverse effects include rare drug- induced hepatitis; ketoconazole is also contraindicated in pregnancy. Itraconazole appears to have fewer side effects than ketoconazole and is used for most of the same indications. Fluconazole also has fewer side effects than ketoconazole that is used for oral or esophageal candidiasis or cryptococcal meningitis. Miconazole is a parenteral imidazole with efficacy in, for example, coccidioidomycosis and several other mycoses, but has side effects including hyperlipidemia or hyponatremia.

The allylamines-thiocarbamates are generally used to treat skin infections. This group includes tolnaftate, naftifine or terbinafine. Another antifungal agent is griseofulvin, a fungistatic agent which is administered orally for fungal infections of skin, hair or nails that do not respond to topical treatment. Other topical agents include ciclopirox or haloprogin. Yet another topical agent is butenafine (Syed et al., J. Dermatol., 25:648-652 (1988)). [Chapter 49 in Goodman and Gilman, The Pharmacological Basis of Therapeutics, 9th ed., McGraw-Hill, New York (1996), pages 1175-1190.]

BPI protein products, a class of products related to bactericidal/permeability-increasing protein (BPI), are described in U.S. Pat. No. 5,627,153 and corresponding International Publication No. WO 95/19179 (PCT/US95/00498), all of which are incorporated by reference herein, to have antifungal activity. BPI-derived peptides with antifungal activity are described in U.S. Pat. No. 5,858,974, which is in turn a continuation-in-part of U.S. application Ser. No. 08/504,841 filed Jul. 20, 1994 and corresponding International Publication Nos. WO 96/08509 (PCT/US95/09262) and WO 97/04008 (PCT/US96/03845), all of which are incorporated by reference herein. Other peptides with antifungal activity are described in U.S. Pat. Nos. 5,652,332 and 5,856,438 [corresponding to International Publication No. WO 95/19372 (PCT/US94/10427)], and in U.S. Pat. Nos.5,763,567 and 5,733,872 [corresponding to International Publication No. WO 94/20532 (PCT/US94/02465)], which is a continuation-in-part of U.S. patent application Ser. No. 08/183,222 filed Jan. 14, 1994, which is a continuation-in-part of U.S. patent application Ser. No. 08/093,202 filed Jul. 15, 1993 [corresponding to International Publication No. WO 94/20128 (PCT/US94/02401)], which is a continuation-in-part of U.S. patent application Ser. No. 08/030,644 filed Mar. 12, 1993, now U.S. Pat. No. 5,348,942, the disclosures of all of which are incorporated herein by reference.

Known antibacterial agents which can be co-administered or combined with the peptide construct, such as XMP.676, according to the invention include antibiotics, which are natural chemical substances of relatively low molecular weight produced by various species of microorganisms, such as bacteria (including Bacillus species), actinomycetes (including Streptomyces) or fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics include (1) the β-lactams, including the penicillins, cephalosporins or monobactams, including those with β-lactamase inhibitors; (2) the aminoglycosides, e.g., gentamicin, tobramycin, netilmycin, or amikacin; (3) the tetracyclines; (4) the sulfonamides and/or trimethoprim; (5) the quinolones or fluoroquinolones, e.g., ciprofloxacin, norfloxacin, ofloxacin, moxifloxacin, trovafloxacin, grepafloxacin, levofloxacin or gatifloxacin (6) vancomycin; (7) the macrolides, which include for example, erythromycin, azithromycin, or clarithromycin; or (8) other antibiotics, e.g., the polymyxins, chloramphenicol, rifampin, the lincosamides, or the oxazolidinones.

Antibiotics accomplish their anti-bacterial effect through several mechanisms of action which include the following general groups: (1) agents acting on the bacterial cell wall such as bacitracin, the cephalosporins, cycloserine, fosfomycin, the penicillins, ristocetin, or vancomycin; (2) agents affecting the cell membrane or exerting a detergent effect, such as colistin, novobiocin or polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, or the macrolide antibiotics such as erythromycin or oleandomycin; (4) agents affecting nucleic acid metabolism, e.g., the fluoroquinolones, actinomycin, ethambutol, 5-fluorocytosine, griseofulvin, rifamycins; or (5) drugs affecting intermediary metabolism, such as the sulfonamides, trimethoprim, or the tuberculostatic agents isoniazid or para-aminosalicylic acid. A new class, oxazolidinones, has recently described, which includes linezolid. Some agents may have more than one primary mechanism of action, especially at high concentrations. In addition, secondary changes in the structure or metabolism of the bacterial cell often occur after the primary effect of the antimicrobial drug.

The penicillins include methicillin, oxacillin, cloxacillin, dicloxacillin or nafcillin, which are generally not affected by the β-lactamase of staphylococci. The penicillins also include amoxicillin or ticarcillin, which are sometimes marketed in combination with the β-lactamase inhibitor clavulanic acid, and ampicillin, which is sometimes marketed in combination with ampicillin.

The cephalosporins include first generation drugs (including cephalothin, cephapirin, cefazolin, cephalexin, cephradine or cefadroxil), second generation drugs (including cefamandole, cefoxitin, ceforanide, cefuroxime, cefuroxime axetil, cefaclor, cefonicid or cefotetan), third generation drugs (including cefotaxime, moxalactam, ceftizoxime, ceftriaxone, cefoperazone or ceftazidime) or fourth generation drugs (including cephamine). Monobactams include imipenem, which is sometimes marketed in combination with cilastin (a compound that inhibits inactivation of imipenem in the kidney), and aztreonam.

The aminoglycosides include amikacin, gentamicin, kanamycin, neomycin, netilmycin, paromomycin or tobramycin. The tetracyclines include tetracycline, chlortetracycline, demeclocycline, doxycycline, methacycline, minocycline or oxytetracycline. The sulfonamides include sulfacytine, sulfadiazine, sulfamethizole, sulfisoxazole, sulfamethoxazole, sulfabenzamide or sulfacetamide. Another inhibitor of dihydrofolate reductase enzyme is trimethoprim, which is also available in combination with sulfamethoxazole (the combination also being known as co-trimoxazole). The fluoroquinolones and quinolones include ciprofloxacin, norfloxacin, ofloxacin, moxifloxacin, trovafloxacin, grepafloxacin, levofloxacin, gatfloxacin, or cinoxacin. The macrolides include erythromycin, clarithromycin, or azithromycin. Lincosamide antibiotics include lincomycin or clindamycin. Vancomycin is a glycopeptide compound with a molecular weight of about 1500. Daptomycin is another recently described antimicrobial compound that is a lipopeptide.

Some drugs, e.g. aminoglycosides, have a small therapeutic window. For example, 2 to 4 μg/ml of gentamicin or tobramycin may be required for inhibition of bacterial growth, but peak concentrations in plasma above 6 to 10 μg/ml may result in ototoxicity or nephrotoxicity. These agents are more difficult to administer because the ratio of toxic to therapeutic concentrations is very low. Antimicrobial agents that have toxic effects on the kidneys and that are also eliminated primarily by the kidneys, such as the aminoglycosides or vancomycin, require particular caution because reduced elimination can lead to increased plasma concentrations, which in turn may cause increased toxicity. Doses of antimicrobial agents that are eliminated by the kidneys must be reduced in patients with impaired renal function. Similarly, dosages of drugs that are metabolized or excreted by the liver, such as erythromycin, chloramphenicol, or clindamycin, must be reduced in patients with decreased hepatic function. In situations where an antimicrobial agent causes toxic effects, the peptide construct, such as XMP.676, may act to reduce the amount of this antimicrobial agent needed to provide the desired clinical effect.

Bacteria acquire resistance to antibiotics through several mechanisms: (1) production of enzymes that destroy or inactivate the antibiotic [Davies, Science, 264:375-381 (1994)]; (2) synthesis of new or altered target sites on or within the cell that are not recognized by the antibiotic [Spratt, Science, 264:388-393 (1994)]; (3) low permeability to antibiotics, which can be reduced even further by altering cell wall proteins, thus restricting access of antibiotics to the bacterial cytoplasmic machinery; (4) reduced intracellular transport of the drug; and (5) increased removal of antibiotics from the cell via membrane-associated pumps [Nikaido, Science, 264:382-387 (1994)].

The susceptibility of a bacterial species to an antibiotic is generally determined by any art recognized microbiological method. A rapid but crude procedure uses commercially available filter paper disks that have been impregnated with a specific quantity of the antibiotic drug. These disks are placed on the surface of agar plates that have been streaked with a culture of the organism being tested, and the plates are observed for zones of growth inhibition. A more accurate technique, the broth dilution susceptibility test, involves preparing test tubes containing serial dilutions of the drug in liquid culture media, then inoculating the organism being tested into the tubes. The lowest concentration of drug that inhibits growth of the bacteria after a suitable period of incubation is reported as the minimum inhibitory concentration.

The resistance or susceptibility of an organism to an antibiotic is determined on the basis of clinical outcome, i.e., whether administration of that antibiotic to a subject infected by that organism will successfully cure the subject. While an organism may literally be susceptible to a high concentration of an antibiotic in vitro, the organism may in fact be resistant to that antibiotic at physiologically realistic concentrations. If the concentration of drug required to inhibit growth of or kill the organism is greater than the concentration that can safely be achieved without toxicity to the subject, the microorganism is considered to be resistant to the antibiotic. To facilitate the identification of antibiotic resistance or susceptibility using in vitro test results, the National Committee for Clinical Laboratory Standards (NCCLS) has formulated standards for antibiotic susceptibility that correlate clinical outcome to in vitro determinations of the minimum inhibitory concentration of antibiotic.

Other aspects and advantages of the present invention will be understood upon consideration of the following illustrative examples. Example 1 addresses preparation and purification of XMP.676. Example 2 addresses in vitro antifungal activity testing of XMP.676. Example 3 addresses in vivo antifungal activity testing of XMP.676. Example 4 addresses testing of XMP.676 for antibacterial activity. Example 5 addresses testing of XMP.676 for endotoxin binding and neutralization. Examples 6, 7 and 8 address testing of other properties of XMP.676, including serum stability, oral availability, and ability to inhibit ATP synthase activity.

### EXAMPLE 1

Preparation of XMP.676

Preparation and purification of Domain III derived peptides, analysis of the properties of these peptides in a number of in vitro and in vivo assays, and therapeutic uses of these peptides are described generally in U.S. Pat. No. 5,858,974 and corresponding International Publication No. WO 97/04008 (PCT/US96/03845). XMP.676 was synthesized and purified generally according to Example 1 of U.S. Pat. No. 5,858,974.

For purity analysis of each newly synthesized peptide, dilute solutions of crude lyophilized peptides were prepared and analyzed on a Beckman 126 HPLC System, using Gold Nouveau software equipped with a 4.6 mm×250 mm, 5 μm particle, 125 Å pore Waters Xterra reverse phase C18 column. The column oven was set to 38° C., the flow rate was 1 mL/minute, and the injection volume was typically 15 to 25 μL. For XMP.676, HPLC was initially performed using 5% acetonitrile, 0.1% trifluoroacetic acid in water as mobile phase A, and B as 80% acetonitrile and 0.065% trifluoroacetic acid in water, or alternatively, 90% acetonitrile, 0.1% trifluoroacetic acid in water as mobile phase B. The elute was monitored spectrophotometrically at 214 nm and 280 nm. Percent purity was calculated from the peak area of the individual peptides.

Selected peptide constructs, such as XMP.676, were purified by high performance liquid chromatography (HPLC), using a Waters Prep LC 2000 Preparative Chromatography System (Waters Corp., Milford, Mass.) equipped with a Delta Pak C18 15 μm, 300 Å cartridge column consisting of a 40×10 mm guard column and a 40×100 mm Prep Pak cartridge. The column was equilibrated in 30% buffer B, where A=5% acetonitrile/0.1% trifluoroacetic acid and B=80% acetonitrile/0.065% trifluoroacetic acid or B=90% acetonitrile/0.1% trifluoroacetic acid. Peptides were dissolved to 20 mg/mL in buffer A and 200-800 mg were applied to the column through the LC pump operating at a flow rate of 17 mL/minute. The bound material was eluted with a gradient of 30-50% B/30 minutes applied at a rate of 17 mL/min. The eluate was monitored at 214 or 280 nm with a Waters 490E Programmable Multiwavelength Detector. Fractions were collected and assayed for the peptide of interest on a Beckman 126 HPLC System, using Gold Nouveau software equipped with a 4.6 mm×250 mm, 5 μm particle, 125 Å pore Waters Xterra reverse phase C18 column, with the column oven set to 38° C. Fractions containing the peptide construct of interest ≧95% were pooled and lyophilized to dryness. The purity of the recovered material was determined with analytical reverse-phase HPLC.

XMP.676 produced in this manner (using a 30-50% B gradient/30 minutes) had about 96% purity, a yield of about 27% and a molecular weight of 1290.7.

### EXAMPLE 2

In Vitro Antifungal Activity

XMP.676 was tested for in vitro antifungal activity in radial diffusion assays against C. albicans SLU-1 generally according to Examples 2 and 3 of U.S. Pat. No. 5,858,974. In the radial diffusion assay, the lowest minimum inhibitory concentration (MIC) of XMP.676 observed against C. albicans SLU#1 was 171 pmol.

XMP.676 was also tested for in vitro antifungal activity against various Candida, Aspergillus, Cryptoccoccus, Trichophyton and Fusarium species in a broth assay generally according to Example 2 of U.S. Pat. No. 5,858,974. Sabouraud's Dextrose broth (SDB) was previously determined to be more optimal than, e.g., RPMI, for antifungal spectrum characterization and susceptibility testing.

Briefly, broth assays were carried out on fungi grown overnight from a single colony to mid-log phase. The cells were washed and assayed in a 96 well format at a final cell concentration of about 2.5×10³ CFU/well. XMP.676 was added in SDB and incubated with the cells for 18 hours at 37 degrees C. The cells were then assayed for growth by OD observation at 595 nm. Alternatively, for Aspergillus cultures, the metabolic indicator dye Alamar Blue (20 μL/well) was added at the same time as XMP.676 and the plate was assayed for Alamar Blue fluorescence at a wavelength of 590 nm (584 nm excitation) following 24 hours of incubation. MIC was defined as the drug concentration where no Alamar Blue fluorescence is observed. For MFC calculations, the contents of the wells were removed after the incubation period and plated on rich media, and MFC was defined as the drug concentration which represents 99-100% killing of the initial innoculum. As shown in Table 1 below, XMP.676 has excellent activity against a range of fungal species.

TABLE 1

| Species | MIC | | MFC |
|---|---|---|---|
| C. albicans ATCC 10231 | 0.5 | μg/mL | not tested |
| C. albicans ATCC 498 | 1.0 | μg/mL | not tested |
| C. albicans #SLU1 | 0.25-16 | μg/mL | 0.5 μg/mL |
| C. parapsilosis | 1.0 | μg/mL | not tested |
| A. fumigatus | 2.0-8 | μg/mL | 4.0 μg/mL |
| C. neoformans | 0.125 | μg/mL | 0.125 μg/mL |
| T. rubrum | 1.0-8 | μg/mL | not tested |
| T. mentagrophytes | 1.0-2.0 | μg/mL | not tested |
| F. solani | 0.25 | μg/mL | 0.25 μg/mL |
| C. tropicalis | 0.125 | μg/mL | not tested |
| C. glabrata | 4.0 | μg/mL | not tested |

The data demonstrate that XMP.676 not only inhibits growth of fungi but also kills fungi. In all cases, the MFC value was equal to or roughly twice the observed MIC value, confirming the fungicidal nature of this compound. XMP.676 is additionally tested with additional fungal species, including C. dubliniensis, C. kruzei, A. niger, F. oxysporum, Sc. hyallinum and P. marneffel.

In addition, checkerboard assays were used to evaluate synergy, additivity or antagonism between XMP.676 and fluconazole, amphotericin B or itraconazole. These assays were carried out on both C. albicans and A. fumigatus. Results from these experiments were used to calculate fractional inhibitory concentration (FIC) values. The results demonstrated that on C. albicans, XMP.676 shows strong additivity or synergy with fluconazole and amphotericin-B, and on A. funigatus, XMP.676 shows additivity with tested concentrations of amphotericin-B and itraconazole. No antagonism was observed with any of the other antifungal agents tested. At lower concentrations of both compounds, additional synergy may be observed.

### EXAMPLE 3

In Vivo Antifungal Activity

XMP.676 was tested for in vivo antifungal activity in mice with systemic C. albicans infection, as measured by effect on mortality, generally according to Example 4 of U.S. Pat. No. 5,858,974.

Briefly described, DBA/2 mice were inoculated with an IV injection of approximately 7×10⁴ CFU C. albicans SLU-1. Desired doses of XMP.676 were prepared from a stock solution of 1 mg/mL XMP.676 in a 0.9% saline solution. Treatment with XMP.676, other antifungal agents or saline via either IV or IP routes was initiated immediately after fungal challenge. Subsequent treatments were administered q.o.d. through day 15. Mortality was recorded daily for 28 days.

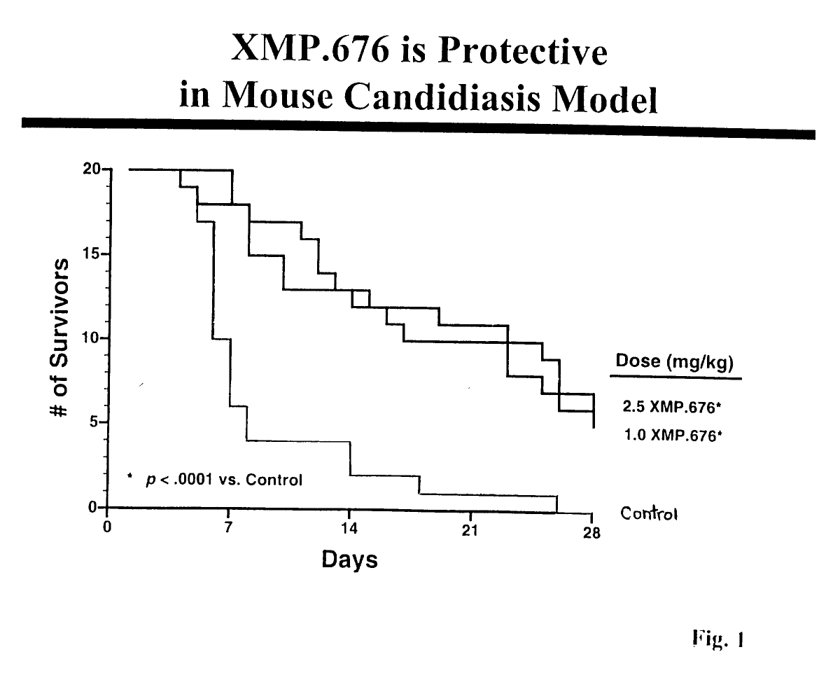

Results of testing XMP.676 alone (without other antifungal agents) in multiple experiments at doses ranging from 0.5 to 5 mg/kg delivered intravenously or intraperitoneally showed a statistically significant reduction in mortality at; a 0.5 mg/kg dose IV (q.o.d. regimen); (p<0.001) reduction in mortality at a 1.0 mg/kg dose IV (q.o.d. regimen) (p=0.019); at a 2.5 mg/kg dose IV in all five of the experiments (p=0.005, p<0.001, p<0.001, p=0.002 and p=0.015) in which that dose was tested (in a q.o.d. regimen) (p<0.05); and at a 5 mg/kg dose IP (q.o.d. or q.d.) (p=0.0001, p=0.012 and p=0.018) in three of four experiments in which that dose was tested. Results of a representative Candida study where XMP.676 was administered at doses of 1.0 and 2.5 mg/kg are displayed in FIG. 1, which shows a reduction in overall mortality and mortality rate in XMP.676-treated mice.

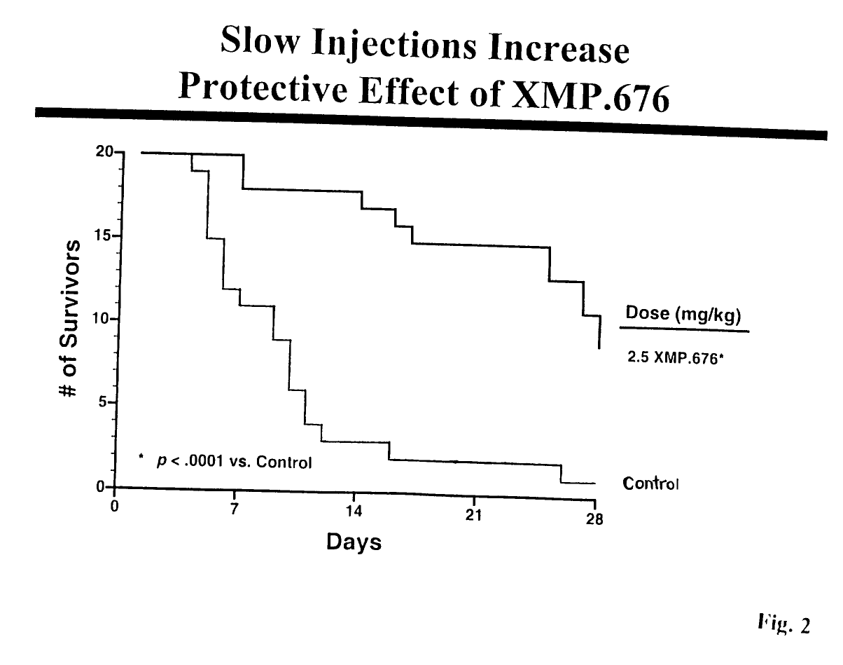

It was observed that a slow injection (approximately 20 seconds), relative to a rapid bolus injection (less than about 5 seconds), resulted in a greater delay in onset of death and greater protection in challenged animals. FIG. 2 shows the results of a representative study where 2.5 mg/kg XMP.676 was administered intravenously via slow injection, and illustrates that slow injection appears to increase the protective effect of XMP.676 relative to rapid injection. It has also been found that intravenous treatment q.o.d. provides greater benefit that intravenous treatment q.d. or b.i.d. Furthermore, intraperitoneal treatment also provided significant protection, suggesting that alternative routes of administration (e.g. subcutaneous injection) may be clinically useful.

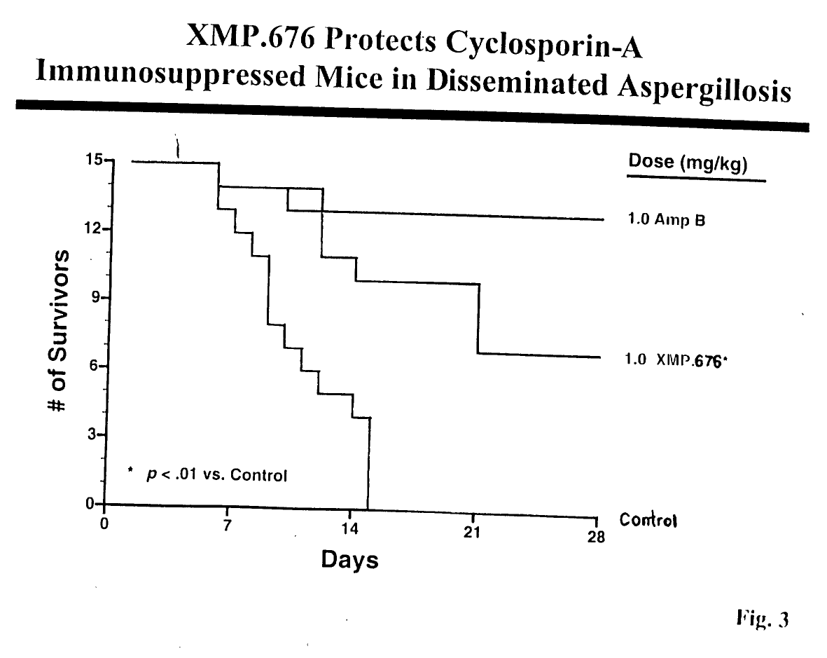

XMP.676 was additionally tested in cyclosporin-A (CSA) immunosuppressed mice systemically infected with about 5×10⁵ CFU Aspergillus fumigatus. Specifically, mice were immunosuppressed by IP injection of 50 mg/kg CSA at day-1. CSA injections continued daily for 9 consecutive days (to day 8). On Day 0, animals received the second CSA injection immediately followed by an inoculum of A. fumigatus in a 0.25 mL volume via a lateral tail injection. Treatment with XMP.676 began one hour following fungal challenge at doses of 0.3, 1.0, 3.0 or 10.0 mg/kg IP, was repeated one day later, and then continued q.o.d. for a total of 7 treatments (Days 0, 1, 3, 5, 7, 9, 11). One group was treated with saline as a control, and another group received 1.0 mg/kg amphotericin B for comparison. Mortality was recorded for 28 days. Results showed that XMP.676 provided a highly statistically significant reduction in mortality (p=0.0003) at a dose of 1.0 mg/kg IP q.o.d.; no reduction in mortality was observed at doses of 3.0 or 10.0 mg/kg. FIG. 3 displays the results of a representative study showing the effect of XMP.676 and amphotericin B in CSA-immunosuppressed mice with disseminated aspergillosis. XMP.676 is also be tested in the absence of immunosuppression with CSA.

A preliminary test of XMP.676 in 5-fluorouracil-treated mice with systemic C. albicans infection (wherein 150 mg/kg 5-FU was administered once intravenously at day-1 and XMP.676 was administered at 5 mg/kg twice daily for 14 days via intraperitoneal injection) did not provide a reduction in mortality compared to saline control.

A preliminary test of XMP.676 in cytarabine-treated rabbits with systemic C. albicans (wherein 30 mg/kg cytarabine was administered intravenously daily for 5 days prior to fungal inoculation on day 0 and every other day thereafter for 10 days). Beginning on the fourth day of the study, immunosuppressed animals were dosed intravenously with 75 mg/kg/day ceftazidime and 15 mg/kg/day vancomycin for the prevention of infection and associated mortality and morbidity. Antibiotic treatment continued every 2 days throughout the course of chemotherapy. XMP.676 administered intravenously as a 30 minute daily infusion at 1.25 mg/kg did not provide a reduction in quantifiable clearance of C. albicans from collected tissues (liver, lung, kidney and spleen).

XMP.676 is also tested in mice in a focal pulmonary infection model using A. fumigatus. In this model, CD-1 mice are immunosuppressed with cortisone acetate and then given an intratracheal injection of 2×10⁵ CFU of A. fumigatus. Treatment with XMP.676, amphotericin B or saline is initiated 3 hours after challenge and then q.d. for 6 days. Mortality is recorded for 14 days and statistical significance is determined.

XMP.676 is also tested in mice infected with Fusarium solani (clinical isolate #99-6. Groups of mice are given 150 mg/kg 5-FU on day-1 to render them neutropenic, infected with 5×10⁶ CFU F. solani IV on day 0, and treated with saline or or XMP.676 at varying doses (e.g. 5 mg/kg IP daily, or 2.5 mg/kg IP twice daily (b.i.d.), or 1 mg/kg IP daily, or 0.5 mg/kg IP b.i.d.) on days 1-7 post-challenge. Mice are observed for 30 days for survival. Mortality is measured and statistical significance is determined.

Increasing doses of various BPI-derived peptides were also administered to healthy DBA/2 mice q.o.d. via intravenous injection into the tail vein, or via intraperitoneal injection, to determine the maximum dose of peptide that could be administered without causing observable symptoms such as respiratory distress and loss of righting reflex. Well tolerated doses given via rapid vs. slow intravenous injection were also compared. For slow injection, injection rate was extended over approximately 20 seconds, while rapid injection occurred in less than 5 seconds. When administered via rapid intravenous injection, doses of XMP.676 higher than 2.5 mg/kg were not well tolerated, but slow injection increased the well tolerated dose two-fold to 5.0 mg/kg. When administered intraperitoneally, XMP.676 was well tolerated at doses up to and including 15.0 mg/kg. Comparison of well tolerated doses to minimum therapeutically effective dose in the mouse model of C. albicans infection shows that XMP.676 exhibits a broad range of therapeutic doses without adverse effects (the ratio for intravenous dosing is about 10:1). XMP.676 was also tested in cytarabine-treated conscious rabbits (wherein 30 mg/kg cytarabine was administered intravenously daily for 5 days prior to administration of XMP.676). XMP.676 administered intravenously as a30 minute single infusion at 2.5 mg/kg to conscious rabbits did not result in any significant adverse events. Higher doses resulted in mortality.

### EXAMPLE 4

In vitro Antibacterial Activity

XMP.676 was tested for activity against various gram-negative and gram-positive bacteria in a Mueller-Hinton broth assay as follows.

For gram-negative bacteria, a single colony was selected to inoculate 5 mL of tryptic soy broth (TSB) for overnight growth. Following overnight incubation, a 1:100 dilution was made into cation-adjusted Mueller Hinton Broth (CAMHB) for growth to mid-logarithmic phase (approximately 3 hours). Cells were washed twice and quantified by OD570 nm determination. (1 OD unit=1.25×10⁹ CFU/mL.) The bacterial suspension was adjusted to a concentration of 2×10⁶/mL in fresh CAMHB.

XMP.676 was two-fold serially diluted in CAMHB from a concentration of 128 μg/mL (64 μg/mL final concentration). Antibiotic controls may include ciprofloxacin, ceftazidime and azlocillin. All compounds were tested in triplicate. Assays were performed in 96-well microtiter plates. Test compounds were in a volume of 100 μL per well followed by the addition of 100 μL of bacterial suspension. The final concentration of bacteria was 1×10⁶/mL with peptides starting from a concentration of 64 μg/mL. The plates were incubated for a period of 24 hours at 37° C. and then read in an ELISA plate reader at a wavelength of 595 nm. Minimum inhibitory activity (MIC) was determined as lowest concentration of peptide construct or other agent which displayed no growth by optical density determination.

For grain-positive bacteria (e.g., S. aureus), the broth assay described above was carried out in the same manner except that 1 OD unit=5×10⁸ CFU/mL.

Results are shown below in Table 2:

TABLE 2

| Species | MIC | |
|---|---|---|
| E.coli J5 | 8-16 | μg/mL |
| E. coli O111: B4 | 16-32 | μg/mL |
| S. marcescens | >64 | μg/mL |
| S. aureus | >64 | μg/mL |

The in vivo activity of XMP.676 against gram-positive or gram-negative bacteria is tested using any procedure or animal model known in the art, including those described in U.S. Pat. Nos. 5,523,288, 5,578,572, 5,627,153, 5,858,974 and 5,646,114, all of which are incorporated herein by reference.

### EXAMPLE 5

Endotoxin Binding and Neutralizing Activity of XMP.676

XMP.676 was tested in an in vitro endotoxin neutralization assay generally according to Example 7 of U.S. Pat. No. 5,858,974, incorporated herein by reference. Briefly, the mouse monocytic cell line RAW 264.7 was induced to proliferation by gamma-interferon. Endotoxin (1 ng/mL) was added together with increasing amounts of XMP.676, and proliferation was quantitated by ³H-thymidine incorporation. RAW cells will not proliferate in the presence of active endotoxin, so thymidine incorporation is directly related to endotoxin neutralization activity. XMP.676 was able to neutralize endotoxin with an EC₅₀ of 3.84±0.461 μg/mL and an IC₅₀ of about 20 μg/mL.

The ability of XMP.676 to neutralize endotoxin in vivo is also tested using any procedure known in the art, including that described in Example 7 of U.S. Pat. No. 5,858,974, incorporated herein by reference.

### EXAMPLE 6

Serum Stability of XMP.676

XMP.676 was also tested for stability using a bioassay generally according to Example 5 of U.S. Pat. No. 5,858,974, wherein the peptide is incubated with SDB, RPMI, mouse serum or human serum for varying amounts of time and the resulting serum-treated product is tested for retention of antifungal activity against C. albicans SLU-1 in a FACScan assay as follows.

A solution of XMP.676 (0.5 mg/mL) was mixed with medium or fresh mouse serum or fresh human serum at a 1:1 ratio to make a 250 μg/mL solution. The resulting mixture was incubated at 37° C. Small sample volumes were removed from the incubation mixture at specific times and placed at −20° C.

Antifungal activity of the peptide/serum (or peptide/media) samples was tested as follows. Mid-log phase C. albicans were suspended in SDB at a concentration of about 1×10⁶ CFU/ml. The suspension was aliquoted in 1 mL portions into polystyrene tubes. The peptide/serum mixture sample were thawed and 50 μL of each sample was aliquoted into each tube and incubated at 37° C. for various times. The cells were then pelleted and resuspended in a solution of 10 μg/mL propidium iodide/PBS and incubated for 5 minutes. Propidium iodide is a fluorescent exclusionary dye that is only taken up by the cell when cell death has occurred. Cells were analyzed for fluorescence on a FACScan instrument using cells treated with 70% ethanol as the control for 100% death, and untreated cells for zero death.

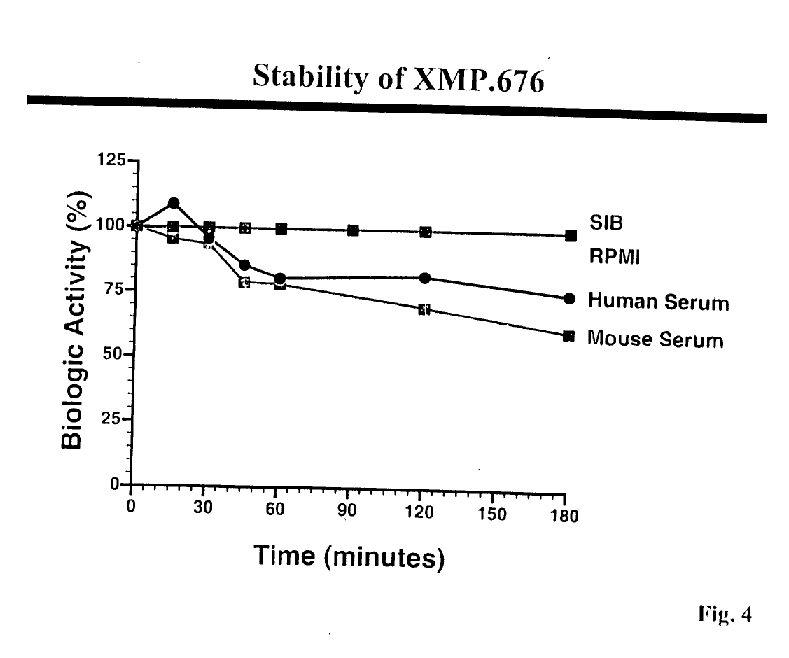

XMP.676 was observed to have excellent stability in all types of microbial media examined and was also quite stable in serum. FIG. 4 shows the results of biological activity stability assays for mouse and human serum and illustrates that XMP.676 retains its activity over time when incubated in either serum. The long biological half life (greater than 180 minutes) of XMP.676 in both mouse and human serum indicates that it should be stable in vivo.

### EXAMPLE 7

Oral Availability of XMP.676

Compounds are screened for oral absorption in in vitro screening assays using CACO-2 and MDCK cells. Cultured monolayers of CACO-2 (Human colon carcinoma) [Audus, K. L., et al. Pharm. Res., 7:435-451 (1990)] or Madin-Derby canine kidney epithelial (MDCK) cells (ATCC Accession No. CCL34) are grown upon collagen-coated, permeable-filter supports (Costar Transwell, Corning Inc., Corning, N.Y.). The cells were grown to confluency and allowed to differentiate. The integrity of the monolayers is determined by measuring the transepithelial resistance. The cells are incubated with XMP.676 on the apical side for 2.5 hours in MDCK screening or 4 hours for CACO-2 screening. The transepithelial transport of the compound is measured by quantitative HPLC analysis of the incubation media on the basolateral side of the cells. Radiolabelled mannitol and cortisone are used as positive controls.

XMP.676 is further tested for activity upon oral administration (oral activity) in a 28-day comparative survival efficacy study in mice systemically infected with Candida albicans. Specifically, male DBA/2 mice (Charles River Laboratories) six weeks of age are dosed with 7.9×10⁴ Candida albicans, SLU-1 in 100 μl intravenously via the tail vein in a single dosage on day 0. Treatment begins immediately thereafter with 400 μl oral gavage of either 0.5% dextrose, or XMP.676 in 0.5% dextrose at various doses (in mg/kg) every other day for a total of eight times. Amphotericin B (Fungizone®) is administered intravenously at 0.5 mg/kg as a positive control every other day for a total of eight times. Twice-day monitoring (once daily on weekends and holidays) for mortality is performed. When orally available compounds are administered, improvements in mortality are observed compared with the dextrose-treated controls.

### EXAMPLE 8

ATP Synthase Inhibitory Activity of XMP.676

The ability of XMP.676 to inhibit ATP synthase is tested using any of the assays described in co-owned, co-pending U.S. Ser. No. 09/543,802 filed Apr. 6, 2000 and corresponding International Publication No. WO 00/______ (Int'l Application No. PCT/US00/09252), all of which are incorporated herein by reference.

For example, ability to inhibit yeast (or other) ATPase, including mitochondrial ATP synthase activity and yeast plasma membrane H⁺/K⁺-ATPase may be determined as follows. In this experiment, membrane preparations of a Saccharomyces cerevisiae mitochondrial ATP synthase and a S. cerevisiae H⁺/K⁺-ATPase are tested.

Yeast plasma and mitochondrial membranes are prepared as follows, in a procedure adapted from Daum et al., J. Biol. Chem., 257:13028-33 (1982). A single S. cerevisiae colony is cultured overnight in 5 mL YPD broth with shaking (approx 250 rpm) at 32° C.; this 5 mL culture is then inoculated into 500 mL of YPD broth and cultured again overnight with shaking at 32° C. The cells are harvested by centrifugation for 5 min. at 3000 rpm in a Sorvall GS-3 rotor at 4° C. The cells are washed with dH₂0 and repelleted. The cells are suspended to 0.5 g wet weight/mL in 0.1 M Tris-HCl, pH 7.5, 10 mM DTT and incubated 10 min. at 32° C. The cells are pelleted, washed with 1.2 M sorbitol, repelleted, and resuspended in 1.2 M sorbitol, 20 mM KPO₄, pH 7.4, to 0.15 g cells/mL.

Zymolyase [Seikagaku America, Rockville, Md.] is added at 5000 mg/g cells, wet weight, and the suspension is incubated at 32° C. for one hour. The cells (now spheroplasts) are harvested by centrifugation for 5 min. at 3000 rpm. The cells are washed twice with 1.2 M sorbitol, repelleting each time. The spheroplasts are homogenized in homogenization buffer [0.6 M mannitol, 10 mM Tris-HCl, pH 7.4, 0.1% BSA, 1 mM PMSF] at 4° C. at a concentration of 0.3 g cells/mL. Homogenization is carried out in a chilled Dounce Homogenizer with a tight fitting seal. The homogenate is diluted with 1 volume of homogenization buffer and centrifuged for 5 min. at 3500 rpm in a JA-20 rotor at 4° C. The supernatant is saved. The pellet is resuspended as before in homogenization buffer and one half the pellet volume of acid-washed glass beads (100-200 nm size) is added. The mixture is vortexed five times in 30 second bursts, followed by 30 seconds on ice after each vortex. The homogenate is centrifuged as before, and the supernatant is combined with the previously saved supernatant.

Membranes are collected by centrifugation at 9000×g. Membranes are resuspended in homogenization buffer and are loaded onto sucrose gradients prepared by layering into a Beckman Ultraclear 1×3.5 tube three layers consisting of 10 ml of 2.25 M sucrose followed by 10 ml of 1.65 M sucrose followed by 10 ml of the 1.1 M sucrose. For each tube, 8.5 ml of the homogenized yeast solution is placed on top of the 1.1 M sucrose layer. Balanced pairs of tubes are centrifuged 14 hours at 80,000×g at 4° C. The mitochondrial membranes should be at the interface between the 1.1 M sucrose and the 1.65 M sucrose layers, while the plasma membrane material should be at the interface of the 1.65 M sucrose and 2.25 M sucrose layers. The 1.1 M sucrose layer is removed from the top and, the interface is removed and saved, then the 1.65 M layer is similarly removed and the next interface is removed and saved. The material from each interface is diluted with 4 volumes of cold water and recentrifuged for 30 minutes at 22,000×g. The pellets are resuspended in 20% glycerol, 1 mM EDTA, 1 mM PMSF, 10 mM Tris-HCl, pH 7.0. Protein concentrations are determined using a Bradford assay.

The effect of a test compound on the enzymic activity of the ATPase in the suspensions is measured by the colorimetric determination of phosphate release, as follows. Assays are conducted in a 96-well plate. Each well contained approximately 1-8 μg (per 100 μL total volume) mitochondrial suspension/microsome suspension in incubation buffer (10 mM MES-Tris, pH 6.5, 25 mM NH₄Cl). Five μL of various dilutions of a test compound are added to give final dilutions ranging from about 0.005 to 50 μg/mL. Each plate contains an Enzyme Blank [test compound and buffer alone, without the ATPase-containing suspension], a Positive Control [ATPase-containing suspension alone, without test compound added], and a Phosphate Standard [ATPase-containing suspension containing 50 nM phosphate (5 μl of 10 mM NaH₂PO₄)].

The 96-well plate is incubated 10 min at 21° C. (room temperature). The reaction is initiated by adding 50 μl of ATP Stock solution [10 mM MES, 15 mM ATP, 15 mM MgSO₄, 25 mM NH₄Cl, 0.05% (w/v) deoxycholate, adjusted to pH 6.5 with Tris base] to each well. The plate is incubated for a total of 15 minutes. Plates are centrifuged in a Beckman J-6M centrifuge for 5 minutes at 1200 rpm. One hundred μL of each supernatant is transferred to a new 96-well plate. One hundred μl of Color Developing Reagent, a combined stop solution and color development reagent, is added [prepared by adding 0.5 g Ascorbic acid to 30 ml H₂O, followed by adding 5 ml 12% Ammonium Molybdate in 12N H₂SO₄ and 5 ml of 10% sodium lauryl sulfate, followed by adjusting total volume to 50 ml with H₂O]. All reagents are added to each row at 30 second intervals to ensure that all samples are incubated for the identical length of time. The OD₆₅₀ₙₘ of each well is determined using a Molecular Devices Vmax Kinetic Microplate Reader (Sunnyvale, Calif.), and the OD value for the Enzyme Blank is subtracted from each value.

It is contemplated that not only XMP.676 but also peptide constructs comprising SEQ ID NO: 1 maybe administered and tested in any of the assays described herein and may be used in any of the therapeutic or other methods described herein.

Numerous modifications and variations of the above-described invention are expected to occur to those of skill in the art. Accordingly, only such limitations as appear in the appended claims should be placed thereon.

What is claimed is:

1. An antifungal peptide construct comprising the sequence set forth in SEQ ID NO: 1.

2. A composition comprising the construct of claim 1 and another antifungal agent.

3. An antifungal peptide having the sequence set forth in SEQ ID NO: 1.

4. A method for treating a subject suffering from fungal infection comprising administering a therapeutically effective amount of the construct of claim 1.

5. The method of claim 4 wherein the construct is administered subcutaneously.

6. The method of claim 4 wherein the construct is administered intravenously.

7. The method of claim 4 wherein the fungal infection involves a fungal species of Candida species, Aspergillus species, Cryptococcus species or Fusarium species.

8. The method of claim 4 further comprising the step of administering one or more additional antifungal agents.

9. A method for killing or inhibiting growth of fungi in vitro comprising contacting the fungi with an effective amount of the construct of claim 1.

10. The method of claim 9 further comprising the step of contacting the fungi with one or more additional antifungal agents.

11. A method for treating a subject suffering from bacterial infection or sequelae thereof comprising administering a therapeutically effective amount of the construct of claim 1.

12. The method of claim 11 further comprising the step of administering one or more additional antibacterial agents.

13. A method for killing or inhibiting growth of bacteria in vitro comprising contacting the bacteria with an effective amount of the construct of claim 1.

14. The method of claim 13 further comprising the step of contacting the bacteria with one or more additional antibacterial agents.

15. Use of an antifungal peptide construct comprising the sequence set forth in SEQ ID NO: 1 in preparation of a medicament for treatment of fungal infection.

16. Use of an antifungal peptide having the sequence set forth in SEQ ID NO: 1 in preparation of a medicament for treatment of bacterial infection.


SEQUENCE LISTING (3 sequences)

SEQ ID NO: 1 - protein, 10 aa, Artificial Sequence
  Description of Artificial Sequence Synthetic Peptide
XXXIQXVXXX

SEQ ID NO: 2 - DNA, 1813 bp, Homo sapiens
  CDS (31)..(1491)
  mat_peptide (124)..(1491)
caggccttgaggttttggcagctctggaggatgagagagaacatggccaggggcccttgcaacgcgccgagatgggtgtccctgatggtgctcgtcgccataggcaccgccgtgacagcggccgtcaaccctggcgtcgtggtcaggatctcccagaagggcctggactacgccagccagcaggggacggccgctctgcagaaggagctgaagaggatcaagattcctgactactcagacagctttaagatcaagcatcttgggaaggggcattatagcttctacagcatggacatccgtgaattccagcttcccagttcccagataagcatggtgcccaatgtgggccttaagttctccatcagcaacgccaatatcaagatcagcgggaaatggaaggcacaaaagagattcttaaaaatgagcggcaattttgacctgagcatagaaggcatgtccatttcggctgatctgaagctgggcagtaaccccacgtcaggcaagcccaccatcacctgctccagctgcagcagccacatcaacagtgtccacgtgcacatctcaaagagcaaagtcgggtggctgatccaactcttccacaaaaaaattgagtctgcgcttcgaaacaagatgaacagccaggtctgcgagaaagtgaccaattctgtatcctccaagctgcaaccttatttccagactctgccagtaatgaccaaaatagattctgtggctggaatcaactatggtctggtggcacctccagcaaccacggctgagaccctggatgtacagatgaagggggagttttacagtgagaaccaccacaatccacctccctttgctccaccagtgatggagtttcccgctgcccatgaccgcatggtatacctgggcctctcagactacttcttcaacacagccgggcttgtataccaagaggctggggtcttgaagatgacccttagagatgacatgattccaaaggagtccaaatttcgactgacaaccaagttctttggaaccttcctacctgaggtggccaagaagtttcccaacatgaagatacagatccatgtctcagcctccaccccgccacacctgtctgtgcagcccaccggccttaccttctaccctgccgtggatgtccaggcctttgccgtcctccccaactcctccctggcttccctcttcctgattggcatgcacacaactggttccatggaggtcagcgccgagtccaacaggcttgttggagagctcaagctggataggctgctcctggaactgaagcactcaaatattggccccttcccggttgaattgctgcaggatatcatgaactacattgtacccattcttgtgctgcccagggttaacgagaaactacagaaaggcttccctctcccgacgccggccagagtccagctctacaacgtagtgcttcagcctcaccagaacttcctgctgttcggtgcagacgttgtctataaatgaaggcaccaggggtgccgggggctgtcagccgcacctgttcctgatgggctgtggggcaccggctgcctttccccagggaatcctctccagatcttaaccaagagccccttgcaaacttcttcgactcagattcagaaatgatctaaacacgaggaaacattattcattggaaaagtgcatggtgtgtattttagggattatgagcttctttcaagggctaaggctgcagagatatttcctccaggaatcgtgtttcaattgtaaccaagaaatttccatttgtgcttcatgaaaaaaaacttctggtttttttcatgtg

SEQ ID NO: 3 - protein, 487 aa, Homo sapiens
MRENMARGPCNAPRWVSLMVLVAIGTAVTAAVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSKLQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQAFAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK